(19) United States
(12) Patent Application Publication
(10) Pub. No.: US 2005/0142518 A1
(43) Pub. Date: Jun. 30, 2005

(54) Arrangement for restoring a periodontosis-induced bone defect

(75) Inventor: Michael Gross, Bremen (DE)

Correspondence Address: ALTERA LAW GROUP, LLC, 6500 CITY WEST PARKWAY, SUITE 100, MINNEAPOLIS, MN, 55344-7704, US

(73) Assignee: BEGO Semados GmbH & Co. KG, Bremen (DE)

(21) Appl. No.: 10/963,083

(22) Filed: Oct. 12, 2004

(30) Foreign Application Priority Data
Oct. 10, 2003 (DE) 103 47 232.0-35

(51) Int. Cl. A61C 5/00; A61F 2/28

(52) U.S. Cl. 433/215; 623/23.58

(57) ABSTRACT

The invention concerns an arrangement for restoring a periodontosis-induced bone defect on the periodontium 2. The arrangement has a bone substitute material 8 which can be introduced in the region of the bone defect, and an elastic membrane 9 which can be arranged around introduced bone substitute material 8 for substantially completely delimiting the bone substitute material 8 from surrounding gingiva 4. The membrane has at least one hole 10 for passing the tooth 1 therethrough.

The invention concerns an arrangement for restoring a periodontosis-induced bone defect on the periodontium.

Various measures for treating periodontosis are known. However what is common to those measures is that they are directed to maintaining the status quo. In other words, specific and targeted bone construction in the region of the periodontosis-damaged periodontium does not take place. This is because no suitable measures, which are simple to implement, for building up bone, are available.

Therefore the object of the invention is to provide means for improved periodontosis treatment.

The invention attains that object with an arrangement for restoring a periodontosis-induced bone defect on the periodontium, which has:
a bone substitute material which can be introduced in the region of the bone defect, and
an elastic membrane which can be arranged around introduced bone substitute material for substantially completely delimiting the bone substitute material from surrounding gingiva,
wherein the membrane has at least one hole for passing a tooth therethrough.

The invention is based on the realisation that an elastic membrane—which can be fitted over a tooth in such a way that the membrane sealingly closes off against the tooth and that the membrane substantially completely covers over in relation to the surrounding gingiva a bone substitute material introduced in the region of the bone defect—makes it possible to provide a treatment method for restoring a bone defect, which is quick and easy and which therefore can also be implemented from costs point of view. In that way periodontosis-damaged bone is not only maintained but re-constructed.

Preferably the hole in the membrane is of a cross-sectional area which, before the tooth is passed through the hole or the membrane is fitted over the tooth, is smaller than a cross-sectional area of the corresponding tooth, at which the membrane reaches its end position. That ensures that the membrane sealingly surrounds the tooth and contamination and impurities are prevented from penetrating into the region of bone regeneration.

Preferably the membrane is resorbable by the human or animal body. That therefore eliminates the need for later operative removal of the membrane.

In a particularly preferred feature the membrane is semipermeable. Preferably a semipermeable membrane is oriented in the arrangement in such a way that fluid issue from the region of the augmented bone defect is possible or fluid can flow away from the part near the bone into the surrounding tissue. It is possible in that way to ensure that serous fluid, in particular wound water, can flow away from the region of the bone defect into the surrounding tissue. In particular in that respect the membrane is of such a nature that cellular components cannot pass through the membrane either from one side thereof or from the other side.

Alternatively a semipermeable membrane is oriented conversely in the arrangement, in other words in such a way that fluids can penetrate into the region of the bone defect from the surrounding tissue. An arrangement of that nature is advantageous when the region of the bone defect and therewith the introduced bone substitute material are to be supplied from the surrounding tissue with nutrients, in particular for accelerating bone reconstruction.

The bone substitute material used is advantageously osteoinductive. Osteoinductive materials induce bone formation—even without contact with the bone which has remained. The use of osteoinductive material is therefore particularly advantageous as it promotes fast bone formation.

Advantageously the bone substitute material is synthetic (manufactured). It therefore manages without organic products or substances which could transmit diseases.

The bone substitute material is preferably in the form of a granular material. Advantageously the granular material grains are mixed with blood in order to form a sticky or tacky material which can be applied in the region of the bone defect.

In another embodiment the bone substitute material is embedded in a gel. In that way it can be easily applied to the bone defect without falling out again.

In a particularly preferred feature the arrangement has fixing means for fixing the membrane to the bone. That provides for fixing and sealing off the membrane in the region of the bone. That ensures that no contact occurs between the bone substitute material and the surrounding gingiva even in the edge regions of the membrane.

Preferably the fixing means are also resorbable by the body.

Further advantageous configurations of the invention are set forth in the appendant claims and the embodiments which are described in greater detail with reference to the accompanying drawing in which:

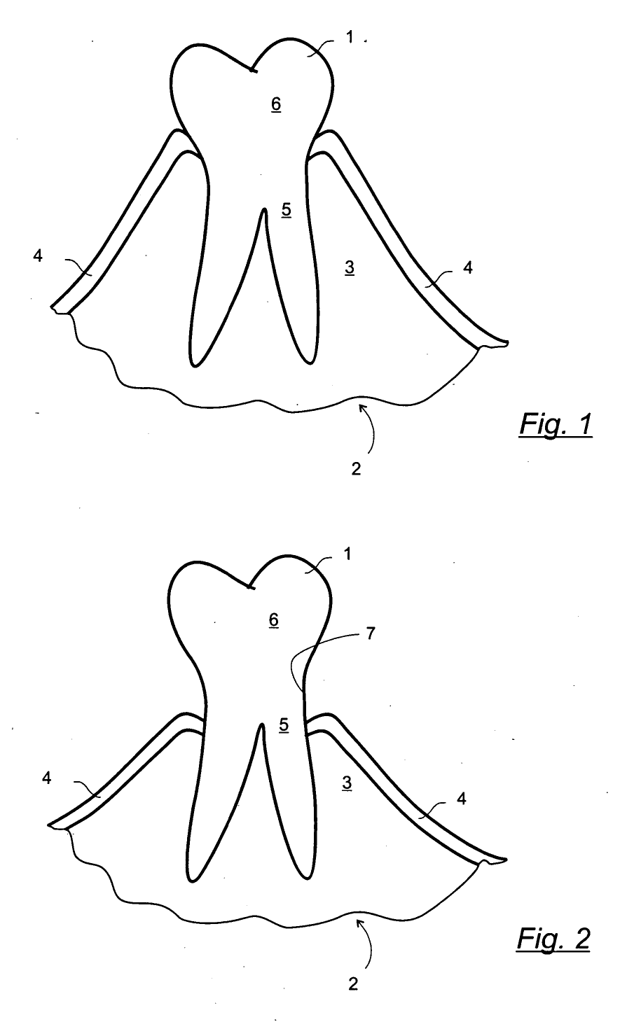

FIG. 1 shows a side view in section through a tooth in a healthy periodontium,

FIG. 2 shows a side view in section through a tooth in a periodontosis-damaged periodontium, in which the gingiva as well as the bone disposed therebeneath have receded,

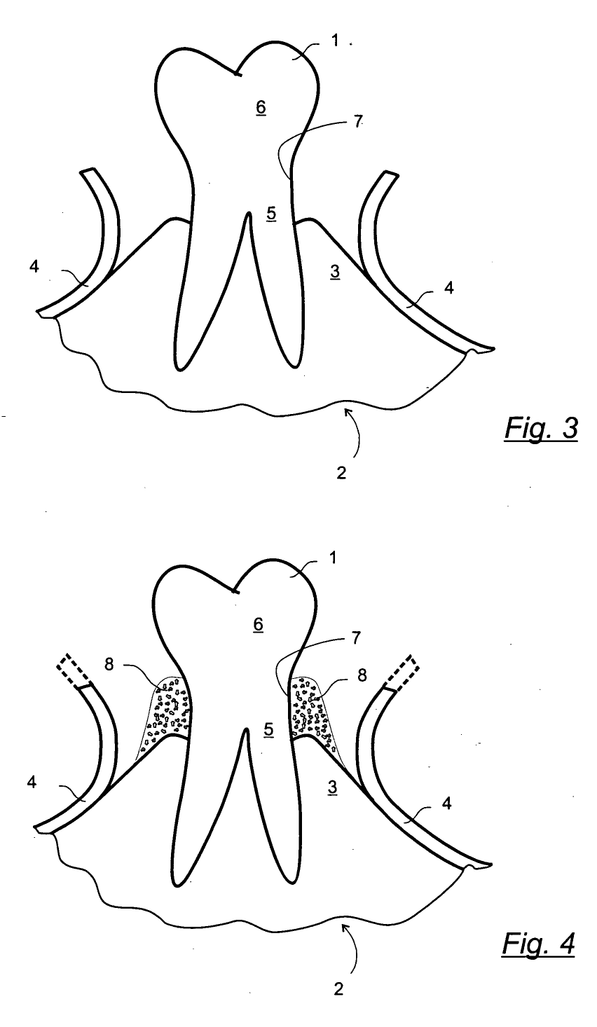

FIG. 3 shows a side view in section of the tooth with periodontosis-damaged periodontium as shown in FIG. 2 with partially separated-off gingiva,

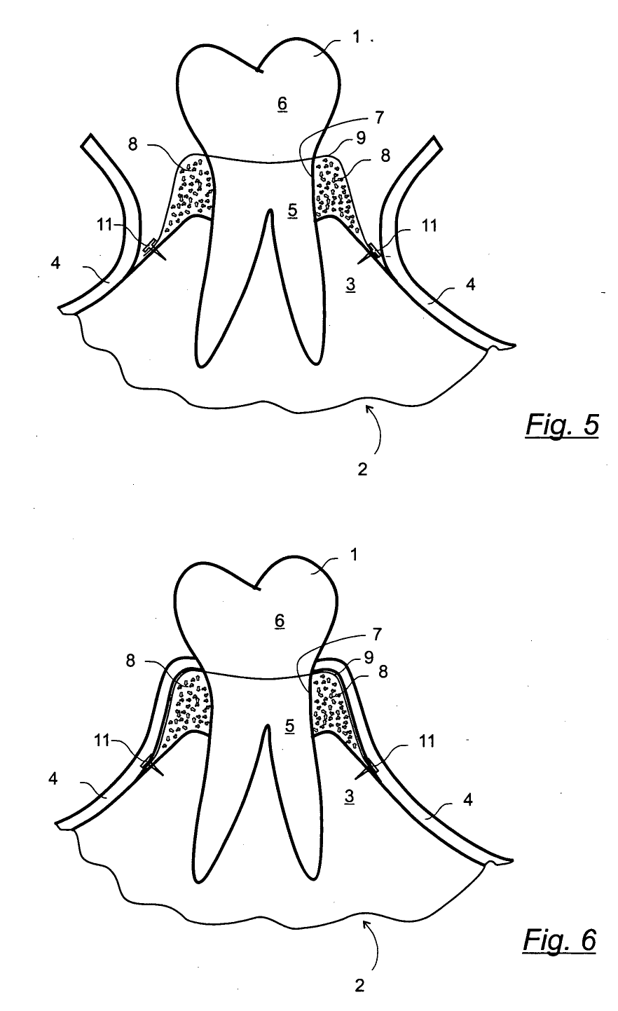

FIG. 4 shows a side view in section of the tooth with periodontosis-damaged periodontium with partially separated-off gingiva as shown in FIG. 3 and bone substitute material applied in the region of a bone defect,

FIG. 5 shows a side view in section of the tooth and periodontium of FIG. 4 with a membrane fitted over the applied bone substitute material,

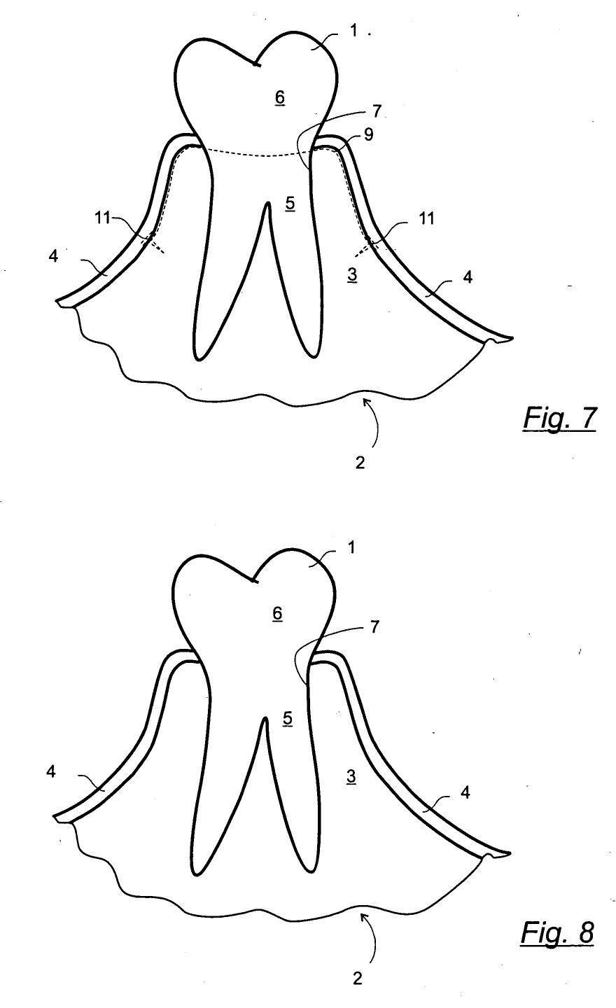

FIG. 6 shows a side view in section of the tooth and periodontium with applied bone substitute material and covering membrane as shown in FIG. 5, the gingiva being laid over the membrane,

FIG. 7 shows a side view in section of the tooth and periodontium of FIG. 6 with reorganised bone defect and partially resorbed membrane and resorbed fixing means,

FIG. 8 shows a side view in section of the tooth and periodontium of FIG. 7 with completely resorbed membrane and completely resorbed fixing means, and

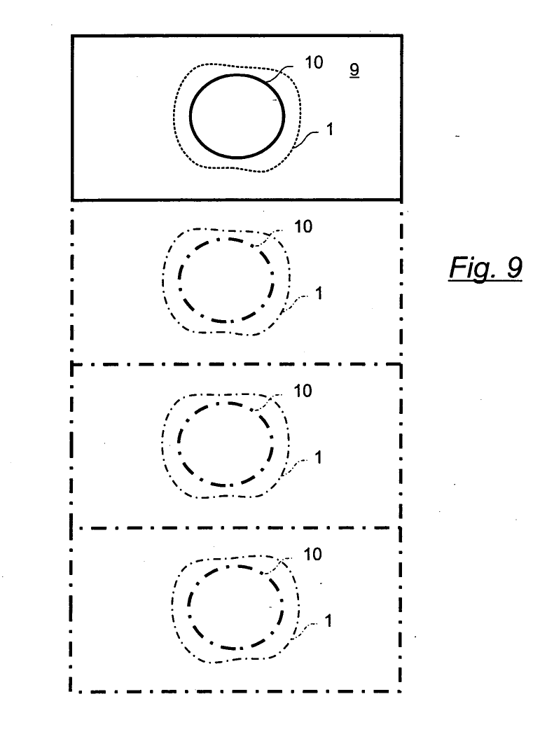

FIG. 9 is a view from above of a membrane for use in an arrangement according to the invention.

FIG. 1 shows a tooth 1 which is grown in place in a healthy periodontium 2. The periodontium includes (inter alia) a bony portion 3 which is covered by a gingiva 4. In this healthy periodontium the root or roots 5 of the tooth 1 is or are completely grown in place and only the tooth crown 6 projects out of the gingiva.

FIG. 2 shows a tooth which has suffered from periodontosis and in which the bone 3 has suffered recession in the region of the neck 7 of the tooth. As the gingiva 4 covering the bone 3 is grown together with the bone 3, the gingiva 4 also suffers recession. The periodontium 2 is damaged. The tooth 1 comes loose. At an advanced stage, finally the tooth falls out.

FIGS. 3 through 8 show an embodiment of a measure according to the invention for the treatment of such periodontosis. As FIG. 3 shows, firstly the gingiva 4, starting from the neck 7 of the tooth, is detached from the bone and the tooth 3, for example by means of cuts with a blade. In that way the operator is provided with space for then carrying out further measures on the bone 3 in the region of the bone defect and along the neck of the tooth or the root surface 7.

FIG. 4 shows two of those measures. In a first measure a bone substitute material 8 is applied along the neck 7 of the tooth in order to restore the portion of the bone which has been lost as a consequence of the periodontosis disease, in the region of the neck 7 of the tooth. That bone substitute material is preferably an osteoinductive bone substitute material, as is described for example in U.S. Pat. No. 6,511,510 B1. The bone substitute material is synthetically manufactured, that is to say it does not have in particular any organic products. Rather, the bone substitute material involves ceramic material, in particular calcium phosphate such as hydroxyapatite with a proportion of between about 70% and 85%, for example substantially 75%, of the bone substitute material. The bone substitute material also has between 15% and 30% of β-tricalcium phosphate.

That bone substitute material also has micropores and macropores. Due to those pores the overall porosity of the material is between 70% and 80%. The micropores are of a size of between about 0.05 μm and 20 μm and the macropores are of a size of between 0.1 mm and 1.5 mm.

The bone substitute material is in the form of a granular material, the granular material grains being of a size of between about 0.25 mm and 3.00 mm, preferably between 0.5 mm and 2.00 mm.

That bone substitute material has osteoinductive properties. In other words, it is bone-forming, more specifically even without contact with the remaining bone being necessary. In contrast thereto osteoconductive materials admittedly promote bone formation, but bone formation takes place only when the material has contact with bone which has remained or which has already re-grown.

Osteoinductive bone substitute material is particularly advantageous as it permits rapid bone formation, in particular rapid vertical bone formation, because bone formation begins in many regions within the applied bone substitute material.

Advantageously the bone substitute material which is present in the form of a granular material is mixed with blood, blood preparations, for example platelet rich plasma, and/or sodium chloride solution (NaCl/physiological saline solution) in order to obtain a spreadable, modellable material. It is only such a material that can be applied in the desired form in the region of the bone defect around the neck 7 of the bone, to the bone 3 which has remained. A bone substitute material which is present purely in the form of a granular material would drop down at unwanted locations during the operation.

Alternatively or additionally the bone substitute material is embedded into a gel. That gel is of such a nature that it can also be applied in plastically shapable and modellable manner in the region of the bone defect.

The second measure shown in FIG. 4 is that of prolonging the gingiva. That is required in order that the gingiva 4 can also be applied again over the bone substitute material 8 applied to the neck 7 of the tooth, in such a way that the bone substitute material 8 is completely covered over, that is to say so that the gingiva 4 extends as far as the tooth crown 6 again. Known methods are available for that purpose, such as for example periosteum slitting.

After preparation of a mucoperiosteum flap, that is to say detachment of gum and bone skin from the bone, the non-stretchable bone skin is carefully severed with a scalpel without injuring further soft tissue. After the severing operation the tissue (mucosa/gingiva) can be stretched and mobilised, thereby affording the desired prolongation effect.

As FIG. 5 shows a rubber-elastic, implantable, in particular tissue-compatible membrane 9 is then fitted over the bone substitute material 8 which is applied along the neck 7 of the tooth on the bone 3 which has remained. The membrane 9 is also at least partially arranged around the tooth 1 and the bone 3. That membrane 9 on the one hand holds the applied bone substitute material in position. On the other hand however the membrane 9 also prevents epithelium from growing over the gingiva 4 into the later bony portion which at that stage still consists of bone substitute material 8.

The membrane 9 preferably comprises a resorbable material. Therefore the membrane does not have to be later removed again, which would necessitate renewed removal of the gingiva 4. Rather the body autonomously dissolves the membrane 9.

The membrane is such that it affords a seam-free seal around the tooth 1. In particular the membrane is formed from a tear-resistant material.

FIG. 9 shows a possible configuration of the membrane 9. The membrane 9 has one or more holes 10 for passing one or more teeth 1 therethrough. That permits the membrane to be fitted over a tooth 1. In order to achieve good sealing integrity for the membrane 9 along the tooth 1, the cross-sectional area circumscribed by the edge of the hole 10 is smaller than the cross-sectional area circumscribed by the contour of the tooth 1 in the region of the end position of the membrane 9.

As is shown in dash-dotted lines in FIG. 9 the membrane can provide a plurality of holes. That is advantageous in particular when it is not just one single tooth that is subjected to periodontosis treatment, but a plurality of teeth are to be treated. In that respect the holes 10 are arranged in such a way that they do not come into contact with each other but a portion of the membrane remains between each two respective adjacent holes 10. That ensures reliable secure sealing integrity for the membrane around each individual tooth.

As shown in FIG. 5 after the membrane 9 has been fitted over a tooth 1 the membrane is fixed to the bone 3 by means of suitable fixing means 11, for example in the manner of a pin, a thumbtack, clip or screw or by means of an adhesive. Fixing means of that kind are either directly introduced into the bone 3 or, if possible, they are arranged in such a way that they completely embrace or clip around the lower region of the introduced bone substitute material 8 and press the membrane 9 against the bone 3.

The fixing means 11 are preferably also of a resorbable material.

As shown in FIG. 6, the gingiva 4 is then laid over the arrangement consisting of bone substitute material 8, membrane 9 and fixing means 11, and sewn along the cuts which were initially made.

As shown in FIG. 7 the bone substitute material is converted completely into bone, in which case new bone is also formed between the bone substitute material grains. In that way, a natural bone is again formed in the region of the original, periodontosis-induced bone defect. At that stage of the bone which has already been reconstructed the membrane 9 and the fixing means 11 dissolve.

As shown in FIG. 8 at the end of the conversion process as bone substitute material the tooth 1 has again had the periodontium 2 grow around same as far as the tooth crown 6. In particular the neck 7 of the tooth is again surrounded by bone 3. Therefore the condition shown in FIG. 8 substantially corresponds to the condition shown in FIG. 1 so that the periodontosis-induced bone defect could be completely restored.

Advantageously, at the beginning of the measures, a splint or brace is fitted to the teeth 1 in order to limit the mobility thereof, that is to say the teeth 1 are immobilised. Splinting is effected for example by fixing a wire or a mesh to the teeth or by a plastic splint which is reinforced with wire and/or mesh and which is fixed to the teeth 1. Alternatively, cast metal splints are used, which can be glued to the teeth. A further alternative involves the use of an individually (lasersintered, milled, cast or molded plastic and/or metal splint.

By virtue of the arrangement according to the invention comprising in particular osteoinductive bone substitute material and an elastic membrane surrounding the bone substitute material, it is possible for bone defects as a consequence of periodontosis to be completely restored. In particular bone regeneration up to the original height is achieved. The teeth are again disposed in a stable periodontium. Tooth loosenings are eliminated.

1. An arrangement for restoring a periodontosis-induced bone defect on the periodontium, which has:
a bone substitute material which can be introduced in the region of the bone defect, and
an elastic membrane which can be arranged around introduced bone substitute material for substantially completely delimiting the bone substitute material from surrounding gingiva,
wherein the membrane has at least one hole for passing a tooth therethrough.

2. An arrangement as set forth in claim 1 wherein the membrane is resorbable by the human or animal body.

3. An arrangement as set forth in claim 1 wherein the membrane is semipermeable.

4. An arrangement as set forth in claim 1 wherein the membrane is tear-resistant and elastically stretchable.

5. An arrangement as set forth in claim 1 wherein the hole is of a cross-sectional area which prior to a tooth being passed therethrough is smaller than a cross-sectional area of the tooth to be passed therethrough.

6. An arrangement as set forth in one of the preceding claim 1 wherein the bone substitute material is osteoinductive.

7. An arrangement as set forth in claim 1 wherein the bone substitute material is synthetic or synthetically manufactured.

8. An arrangement as set forth in claim 7 wherein the bone substitute material has ceramic material, in particular calcium phosphate, in particular hydroxyapatite.

9. An arrangement as set forth in claim 8 wherein the bone substitute material has between 70% and 85% of hydroxyapatite.

10. An arrangement as set forth in claim 7 wherein the bone substitute material has between 15% and 30% of β-tricalcium phosphate.

11. An arrangement as set forth in claim 1 wherein the bone substitute material has micropores and macropores and an overall porosity of between 70% and 80%, wherein the micropores are of a size of between 0.05 μm and 20 μm and the macropores are of a size of between 0.1 mm and 1.5 mm.

12. An arrangement as set forth in claim 1 wherein the bone substitute material is in the form of a granular material.

13. An arrangement as set forth in claim 12 wherein the granular material grains are of a size of between 0.25 mm and 3.00 mm, preferably between 0.5 mm and 2.00 mm.

14. An arrangement as set forth in claim 11 wherein the bone substitute material is mixed with blood, blood preparations and/or sodium chloride solution.

15. An arrangement as set forth claim 1 wherein the bone substitute material is embedded in a gel.

16. An arrangement as set forth in claim 1 which further has fixing means for fixing the membrane to the bone.

17. An arrangement as set forth in claim 16 wherein the fixing means are in the manner of a pin, a thumbtack and/or a clip or adhesive.

18. An arrangement as set forth in claim 16 wherein the fixing means are resorbable by the human or animal body.

19. An arrangement as set forth in claim 1 which further has a bracing splint for immobilizing loose teeth.

20. A method of restoring a periodontosis-induced bone defect of a bone of a periodontium of a tooth, including a gingiva, comprising the steps:
releasing the gingiva,
applying a bone substitute material along the neck of the tooth,
fitting an elastic membrane at least partially around the bone and around the tooth in such a way that the bone substitute material is held in position, and
laying the gingiva over the membrane.

21. A method as set forth in claim 20 additionally with the step of:
fixing the membrane to the bone.

22. A method as set forth in claim 21 wherein fixing means which are resorbable by the human or animal body are used for fixing purposes.

23. A method as set forth in claim 20 wherein application of a bone substitute material is the application of osteoinductive bone substitute material.

24. A method as set forth in claim 20 wherein the bone substitute material is embedded in a gel.

25. A method as set forth in claim 20 additionally comprising the step of:
prolonging the gingiva by periosteum slitting.

26. A method as set forth in claim 20 wherein the membrane is resorbable by the human or animal body.
